(19) United States
(12) Patent Application Publication
(10) Pub. No.: US 2022/0156640 A1
(43) Pub. Date: May 19, 2022

(54) SYSTEM FOR ACOUSTIC IDENTIFICATION OF OBSTRUCTION TYPES IN SLEEP APNOEA, AND CORRESPONDING METHOD

(71) Applicant: DIAMETOS GMBH, Potsdam (DE)

(72) Inventor: Christoph JANOTT, Berlin (DE)

(21) Appl. No.: 17/436,623

(22) PCT Filed: Feb. 24, 2020
(86) PCT No.: PCT/EP2020/054789

§ 371 Date: Sep. 6, 2021

(30) Foreign Application Priority Data
Mar. 7, 2019 (DE) 10 2019 105 762.0

(51) Int. Cl. G06N 20/00; G16H 50/70; G16H 50/20; A61B 5/00; A61B 7/00

(52) U.S. Cl. CPC G06N 20/00; G16H 50/70; G16H 50/20; A61B 5/742; A61B 7/003; A61B 5/7203; A61B 5/7267; A61B 5/4818

(57) ABSTRACT

The present invention relates to a classification system (1) for microprocessor-assisted identification of obstruction types (O1-O4) in sleep apnoea by means of appropriate classification of a snoring-noise signal (Au) to be analysed. The system comprises: a) an input interface for each snoring-noise signal (Au); b) a first classifier (K1) which can be trained such that it identifies and outputs the most probable type of snoring-noise origin (S1-S4) for a particular snoring-noise signal (Au); c) a second classifier (K2) which can be trained such that it identifies and outputs the most probable mouth position (M1-M2) for a particular snoring-noise signal (Au); and d) a third classifier (K3) or linkage matrix, which is designed to identify and output the most probable obstruction type (O1-O4) from the snoring-noise signal (Au) to be analysed, the determined type of snoring-noise origin (S1-S4) and the mouth position (M1-M2) determined therefor.

The present invention relates to a system for microprocessor-supported identification of obstruction types in a sleep apnoea by corresponding classification of a snoring-noise signal to be examined.

Snoring is defined as a nocturnal breathing noise caused by vibrations of soft tissue in the upper respiratory tract. There are various definitions of the term “snoring”, in many of which snoring is a vibration of the tissue of the upper respiratory tract caused by an air stream, leading to a tonal portion in the resulting snoring noise. There is, however, no clear distinction between “snoring” and “loud breathing”. In the following, the term “snoring” can also be understood to be a general type of noise which may also contain breathing noises without a significant tonal portion.

A snoring-noise signal is understood to be an acoustic signal which has been recorded, for instance, by a microphone and has been converted into an electrical signal. The snoring-noise signal may also contain or include additional information in the form of indicators or labels which transmit one or more additional pieces of information on the acoustic snoring-noise signal, such as, for instance, a snoring-noise place of origin, a snoring-noise type of origin, a mouth position, a time of day, a patient's name, a patient's weight and a sleeping position.

Obstructive sleep apnoea is understood to be a condition in which nocturnal pauses in breathing occur due to closures and respectively obstructions of the upper respiratory tract so called respiratory tract obstructions. Depending on the number of obstructive pauses in breathing per hour, various severity levels of obstructive sleep apnoea are distinguished. For obstructive sleep apnoea, snoring is a frequent accompanying symptom. Snoring and obstructive sleep apnoea are sleep-related breathing disorders. In the following, obstructive sleep apnoea is simply called sleep apnoea.

The snoring noises and obstructions of the respiratory tract are produced in different places of the upper respiratory tract and in various ways. The various ways can be determined by a respective orientation and type of vibration or constriction, which may be, for instance, circular or shaped as a lateral slot. Consequently, there are different types of origins of snoring noises which are anatomically connected to the various sites and types of obstruction. In other words, the type of origin of the snoring noise is defined by the snoring-noise place of origin, the orientation and type of vibration or a combination thereof. Analogous, the type of obstruction is defined by the site of obstruction, the orientation of the obstruction or a combination thereof. The different types of origins of snoring noises can be classified as follows:
A) an anterior-posterior vibration of the soft palate;
   C) a circular constriction of the respiratory tract in the velopharynx or oropharynx;
L) a lateral vibration of the soft tissue in the area of the oropharynx;
T) a constriction in the area of the tongue base; and/or
E) a vibration or constriction in the area of the epiglottis.

In the pharynx, the snoring noise can be generated, for instance, in the area of the soft palate with the uvula, in the area of the palatal tonsils, in the area of the tongue base and on the level of the epiglottis. Numerous classification systems have been described in the art, with the aim of establishing a standardized designation of constriction sites and obstruction types in clinical practice. FIG. 2 shows a lateral sectional view of a patient's head with the snoring-noise origin sites in neck, nose and throat with the areas velopharynx (V), oropharynx (O), tongue base (T) and epiglottis (E).

The diagnostics of sleep-related breathing disorders comprise, according to most medical guidelines, a measurement of the frequency of apnoea caused by obstructions and breathing difficulties caused by constrictions of the respiratory tract (hypopnoea) in natural sleep, with the measurements being performed both as polysomnography in the sleeping lab and as polygraphy and cardiorespiratory screening in domestic environments. These measurements, however, do not provide unequivocal information on the site of a constriction or obstruction. Depending on the site and shape of the constriction or obstruction, various therapeutic measures can be taken accordingly. In addition to knowing the severity of the obstructive sleep apnoea, which is determined by the frequency and length of the breathing pauses, it is very important for targeted therapeutic treatment of obstructive sleep apnoea to know the respective obstruction type. It is also of great importance to know the type of origin of the snoring noise to treat snoring in a targeted manner.

One known solution for determining the site of origin of the snoring noise and of the obstruction site is, for instance, nocturnal manometry, in which a thin catheter, of a few millimeters in diameter, equipped with several sensors arranged in series, is introduced through the nose into the upper respiratory tract of the patient. Thus, the pressure conditions during natural sleep can be measured during the night in several positions of the upper respiratory tract. One advantage of this method is continuous measurement during natural sleep. A disadvantage is that not every patient tolerates a catheter in his upper respiratory tract throughout an entire night. Also, no information can be gained on the type and shape of the obstruction.

Another well-known method of determining the sites and types of obstruction in patients is medication-induced sleep video endoscopy. With this method, the patient is sedated by an anesthetist so as to produce an artificial sleep. Then the upper respiratory tract is monitored by means of a flexible endoscope introduced through the nose. Thus, the site, shape and type of the obstructions can be visualized and determined. Disadvantages of this method are stress on the patient caused by sedating medication as well as great effort in terms of staff and apparatuses and high connected costs. During medication-induced sleep video endoscopy, frequently audio recordings of the snoring noises have been made simultaneously with video recordings of the endoscopic examinations, from which in retrospect snoring-noise signals with information on the respective types of origin of snoring noises and/or on the respective types of obstruction can be extracted. There is a certain correlation between the type of origin of the snoring noise and the type of obstruction; however, the type of obstruction cannot be deduced unequivocally from the type of origin of snoring noise. Also, in the large majority of cases, obstructions and snoring noises occur within a certain temporal relationship, but not synchronously in time.

It is known that the mouth position during sleep has a significant influence on the quality of sleep and on other conditions, such as, for example, teeth disorders and on the probability of occurrence and type of an obstruction of the respiratory tract. The mouth position is substantially determined by the position of the mandible and the lips. In the simplest case, two situations are distinguished: mouth closed (e. g. lip closure) and mouth opened. Alternatively, the mouth position can be described in more detail with differentiating definitions.

During medication-induced sleep video endoscopy, open and closed mouth positions are forced, for instance by manual movement of the patient's mandible by the examiner, and the influence of the mouth position on the type of obstruction is examined in this manner. From the audio recordings and endoscopic video recordings of sleep video endoscopy, however, the mouth position at the time where snoring noises occurred cannot be recognized. Information on the mouth position at the time of the occurrence of snoring noises during sleep video endoscopic examinations is not available in structured form, either.

For determining the mouth position during natural sleep, various methods, for instance, video-based methods, are known. For this purpose, respective snoring-noise signals have been recorded for which the corresponding mouth position can be extracted as additional information, can be determined and recorded as an indicator. Here, it is to be noted that for determining the mouth position, a video-based determination requires great time effort and, as an alternative method, determination by means of sensors attached to the patient's head disturbs the quality of sleep. On the other hand, additional snoring-noise signals obtained in this manner, together with the corresponding mouth position, are available and accordingly helpful.

WO2017/009081A1 discloses a device and a method for snoring-noise identification in the upper respiratory tract where breathing takes place through a tube with preferably two offset microphone sensors. By means of the sensor signals of the two microphone sensors, obstructions of the upper respiratory tract can be recognized during inhalation and exhalation.

WO2012/155257A1 discloses a device and a method for diagnosing noises and rhythms of the respiratory tract, where during breathing or sleep, a microphone sensor is positioned in front of the patient's nose.

DE102006008844A1 discloses a method of detecting noises of the respiratory tract, where a sensor is positioned in front of a nostril or introduced into it, and its sensor signal is accordingly evaluated.

For clarity, it should be noted here that the respective snoring-noise signal can comprise the one or more additional pieces of information or that they can be attached to it, the one or more additional pieces of information being associated with the acoustic snoring-noise signal as indicators or labels. These one or more additional pieces of information or indicators can be, for example, the site of origin of snoring noise, the type of origin of snoring noise, the mouth position and/or additional patient parameters as indicated above. The indicators, frequently also called labels, can be modulated, for example, onto the snoring-noise signal itself or contained in it in an encoded manner or can be recorded in a second signal track or file, or can be recorded in writing.

The object of the invention for eliminating drawbacks from the state of the art therefore consists in the provision of a system for automatic and, if possible, significant recognition of the type of obstruction most probable in each case from a snoring-noise signal to be examined.

The object indicated above is achieved by a device according to the features of the independent Claim 1. Other advantageous embodiments of the invention are indicated in the dependent Claims.

According to the invention, a classification system for microprocessor-supported recognition of the types of obstruction of a sleep apnoea by the corresponding classification of a snoring-noise signal to be examined is introduced, comprising:
an input interface for the respective snoring-noise signal;
a first classifier adapted to learn in a training mode, if a first plurality of snoring-noise signals with a respective type of origin of snoring noise is input, such that in an identification mode, it identifies and outputs the most probable of a group of predefined types snoring-noise origin for a particular snoring-noise signal;
a second classifier adapted to learn in a training mode, if a second plurality of snoring-noise signals with a respective mouth position is input, such that in an identification mode, it identifies and outputs the most probable of a group of predefined mouth positions for a particular snoring-noise signal;
a third classifier designed to identify, in an identification mode, from the input of the type of snoring-noise origin determined by the first classifier and the mouth position determined by the second classifier, the most probable of a group of predefined obstruction types, and output the same as obstruction type signal;
an output interface for the obstruction type signal to be indicated.

Preferably, the third classifier is also adapted to learn in a training mode, when the type of snoring-noise origin identified by the first classifier, the mouth position identified by the second classifier and a type of obstruction are input, in such a way that in the identification mode, it identifies the type of obstruction input during training for the respective type of snoring-noise origin and mouth position as the most probable type of obstruction. For purposes of clarity, it is noted that the person skilled in the art knows what is intended by “learning” in the field of classifiers.

The advantages of the invention consist especially in the fact that for training the first and the second classifier, a plurality of snoring-noise signals can be used which only contain either the type of snoring-noise origin or the mouth position as additional information in addition to a merely acoustic component of the snoring-noise signal. The information can generally be encoded in the snoring-noise signal itself as indicator or label or modulated onto it, recorded in a separate signal track or file, or can be attached to the snoring-noise signal, for instance as a written label. Thus, the first classifier can be trained by means of a large amount of snoring-noise signals already available in the state of the art, which only comprise the type of snoring-noise origin as a label, without training the second classifier and/or the third classifier erroneously. In the same manner, the second classifier can be trained by means of another large amount of snoring-noise signals already available in the state of the art, which only comprise the mouth position as a label, without training the first classifier and/or the third classifier erroneously. The classification system according to the invention therefore does not require an entirely new recording of the snoring-noise signals recorded in combination with the two indicators «type of snoring-noise origin» and «mouth position». Since according to the present invention, the existing snoring-noise signals can be used with only one or the other indicator for training the first and the second classifier, a great advantage in terms of costs and effort is achieved; nevertheless, during identification of the type of obstruction starting from the snoring-noise signal to be examined, both indicators “type of snoring-noise origin” and “mouth position” are taken into account. The use of these two indicators in combination substantially increases the precision in correctly identifying the respective type of obstruction, and/or a faulty identification becomes less probable, compared with identification of the type of obstruction from the snoring-noise signal to be examined with the type of snoring-noise origin as the only indicator.

Preferably, the first classifier and the second classifier are configured such that the training of the first and the second classifier, respectively, can be performed separately with a plurality of snoring-noise signals, with the first classifier being trainable independently of the mouth position and the second classifier being trainable independently of the type of snoring-noise origin. Preferably, the training of the first classifier can take place with a time shift with respect to the training of the second classifier. Alternatively preferably, training of the first classifier can take place simultaneously with training of the second classifier. For clarity, the plurality of snoring-noise signals can comprise a series of snoring-noise signals to which the type of snoring-noise origin, and no mouth position, is assigned as indicator; and a different series of snoring-noise signals to which the mouth position, and no type of snoring-noise origin, is assigned as indicator.

For clarity, the terms “indicator” and “label” are here understood as synonyms. The training of the classifier can also be called “learning”. By “training” or “training mode” of the respective classifier with the respective snoring-noise signal with the at least one indicator, it is intended that the respective classifier changes during this process such that in the identification mode, it can better identify the at least one indicator from the snoring-noise signal, preferably in the average in case of many trained snoring-noise signals with several indicators, as is known to the person skilled in the art. The person skilled in the art is aware that, the more different snoring-noise signals are used for training the respective classifier, the higher the identification rate rises.

Of course, the first and the second classifier are preferably also configured to be trainable together with another plurality of snoring-noise signals which include both the type of snoring-noise origin and the mouth position as assigned indicators. In this manner, the first plurality, the second plurality and/or the additional plurality of different snoring-noise signals can be employed for training the first and the second classifier.

The person skilled in the art furthermore knows that the first, the second and the third classifier may technically be part of only one classifier; in this case, training and/or identification of the type of snoring-noise origin and mouth positions and/or of the obstruction types may take place by means of partial classifiers. In addition, the person skilled in the art knows that the information on type of snoring-noise origin and mouth position may alternatively be provided by a series of labels or indicators each of which characterizes a combination of type of snoring-noise origin and mouth position. Preferably, the respective label or indicator may include one or more pieces of information on the snoring-noise signal, for instance the type of snoring-noise origin, the mouth position, the sleeping time and the like.

An improved identification of the types of snoring-noise origin and of the mouth positions from the snoring-noise signal leads to an improved identification of the type of obstruction.

For purposes of clarity, it is noted here that the first classifier is a learning classifier trainable in a training or learning mode by the first series of snoring-noise signals such that subsequently, in an identification mode, it can identify the respective type of snoring-noise origin with highest probability from the respective snoring-noise signals. In the same way, the second classifier is a learning classifier trainable in a training or learning mode by the second series of snoring-noise signals such that subsequently, in the identification mode, it can identify the respective mouth position with highest probability from the respective snoring-noise signals.

The person skilled in the art knows that the respective classifier is preferably optimized to recognize predefined characteristics.

Training and identification of snoring-noise signals by the respective classifiers may take place subsequently, simultaneously or with an overlap in time.

Preferably, the first classifier is adapted to identify in the identification mode the respective type of snoring-noise origin with a corresponding probability, to indicate it and to forward it to the third classifier. The third classifier then evaluates the probability of the respective type of snoring-noise origin in combination with the respective mouth position and, if so desired, additional snoring or patient data. Preferably, the first classifier classifies the snoring-noise signal in a vector of types of snoring-noise origins, the type of snoring-noise origin or class of type of snoring-noise origin being output as a respective probability. This increases the evaluation possibilities and combinatorial analysis, fed with probabilities, between the identified type of snoring-noise origin and identified mouth position for the third classifier.

Preferably, the second classifier is adapted to identify in the identification mode the respective mouth position with a corresponding probability, to indicate it and to forward it to the third classifier. The third classifier then evaluates the probability of the respective mouth position in combination with the respective type of snoring-noise origin and, if so desired, additional snoring or patient data. Preferably, the second classifier classifies the snoring-noise signal in a vector of mouth positions, each mouth position or class of mouth position being output as a respective probability.

Preferably, the third classifier is adapted to identify in the identification mode the respective obstruction type with a corresponding probability, to indicate it and to forward it to the output interface. The output interface then provides the type of obstruction to a notification feature; the output interface can be any of the interfaces known to be suitable for this purpose, for instance an electrical interface or a wireless interface, for instance to a smart phone or a display of a PC. The output interface may also be connected to the internet so that evaluation and display can take place at different locations.

The individual components of the system described may also be spatially separated. Preferably, the respective information will then be transferred between the system components via suitable interfaces which may for instance be electrical interfaces or wireless interfaces. This information may also be transmitted via the internet.

Preferably, the third classifier is adapted to record, in addition to the type of snoring-noise origin and the mouth position, the additional snoring or patient data of the snoring person via an input interface and to take them into account in the training mode and/or the identification mode when classifying the type of obstruction. The additional snoring or patient data may be parameters or parameter signals for even better determining the type of obstruction which is most probable. “Better” here means “with a higher hit rate”.

Preferably, the snoring or patient data comprise at least one of the following parameters: sex, body mass index, apnoea hypopnoea index, size of the tonsils, size of the tongue, Friedman score, time of snoring, time of sleep and/or patient weight.

Snoring events and obstruction events may occur together, but this is not necessarily always the case. For purposes of clarity, it is noted that the label of the obstruction type which is used, together with the respective information on the type of snoring-noise origin and the mouth position, for training the third classifier, may also designate the type of obstruction in case of obstruction events, which have occurred in a certain temporal connection, but not simultaneously with the respective snoring event connected with a specific patient.

Preferably, the first classifier is based on one of the following methods of machine learning or a classification: Support Vector Machine—SVM—, Naive-Bayes System, Least Mean Square Method, k-Nearest Neighbours Method—k-NN—, Linear Discriminant Analysis—LDA—, Random Forests Method—RF—, Extreme Learning Machine—ELM—, Multilayer Perceptron—MLP—, Deep Neural Network—DNN—, logistic regression. Other methods known from the state of the art are conceivable as well and can be applied herein.

Preferably, the second classifier is based on one of the following methods of machine learning or a classification: Support Vector Machine—SVM—, Naive-Bayes System, Least Mean Square Method, k-Nearest Neighbours Method—k-NN—, Linear Discriminant Analysis—LDA—, Random Forests Method—RF—, Extreme Learning Machine—ELM—, Multilayer Perceptron—MLP—, Deep Neural Network—DNN—, logistic regression. Other methods known from the state of the art are conceivable as well and can be applied herein.

Preferably, the third classifier is based on one of the following methods of machine learning or a classification: Support Vector Machine—SVM—, Naive-Bayes System, Least Mean Square Method, k-Nearest Neighbours Method—k-NN—, Linear Discriminant Analysis—LDA—, Random Forests Method—RF—, Extreme Learning Machine—ELM—, Multilayer Perceptron—MLP—, Deep Neural Network—DNN—, logistic regression. Other methods known from the state of the art are conceivable as well and can be applied herein.

It is also possible to assign to the first and/or the second classifier the snoring or patient data or part thereof which the respective first and/or second classifier can evaluate or take into account when classifying the snoring-noise signal. For instance, the features of patient sex, body mass index, apnoea hypopnoea index, tonsil size, tongue size, Friedman score, time of snoring and/or duration of sleep can be assigned to the classifier.

Alternatively preferably, the third classifier can be based on a matrix probability calculation of a first input vector from the types of snoring-noise origin and from at least one second input vector of the mouth positions, whose summary probabilities result in the various obstruction types and their probabilities.

For purposes of clarity, by the first group of types of snoring-noise origin, a first group of first classes of types of snoring-noise origin is intended.

Preferably, the group of types of snoring-noise origin comprises the following classes: velopharynx (V), oropharynx (O), tongue base area (T) and/or epiglottis area (E). Naturally, other classes or sites or types of snoring-noise origin are conceivable as well.

For purposes of clarity, by the group of mouth positions, preferably the group of classes of mouth positions is understood. Preferably, the group of mouth positions comprises the mouth positions “mouth open” and “mouth closed”; other mouth positions and intermediate positions are naturally conceivable as well.

For clarity, by the group of obstruction types, preferably the group of classes of obstruction types is understood.

For clarity, the type of noise generation describes, in addition or instead of the location of noise generation, an orientation and/or shape of the vibration which is herein designated as “type of snoring-noise origin”.

For purposes of clarity, the indicators, also called labels, are preferably determined based on an objective reference value (Ground Truth), preferably determined by observation of the endoscopic image of a medication-induced sleep video endoscopy by by an experienced observer at the time of occurrence of the respective snoring event. Indicators or labels for the mouth position are preferably obtained by observation of the patient during examination, evaluation of video recordings of the mouth area of the patient during the examination or recording of sensor data via the air stream through mouth and nose or other sensors and by documenting the mouth position over the time of recording of the snoring-noise signal.

From a sufficient number of characteristic vectors and training data, the classifier, which is a machine classifier, generates at least one model. If a characteristic vector without a label is fed into the model, it will output a result value. The result value contains information on the most probable class to which the snoring event on which the characteristic vector is based pertains. In an alternative embodiment, the model additionally outputs information on the probability with which the snoring event pertains to the most probable class; alternatively in addition on the probability of belonging to the other classes, as described above. Preferably, the output classes correspond to the classes of the label used for training.

Preferred embodiments of the present invention are described in the following figures and in a detailed description; however, they are not intended to limit the present invention thereto:

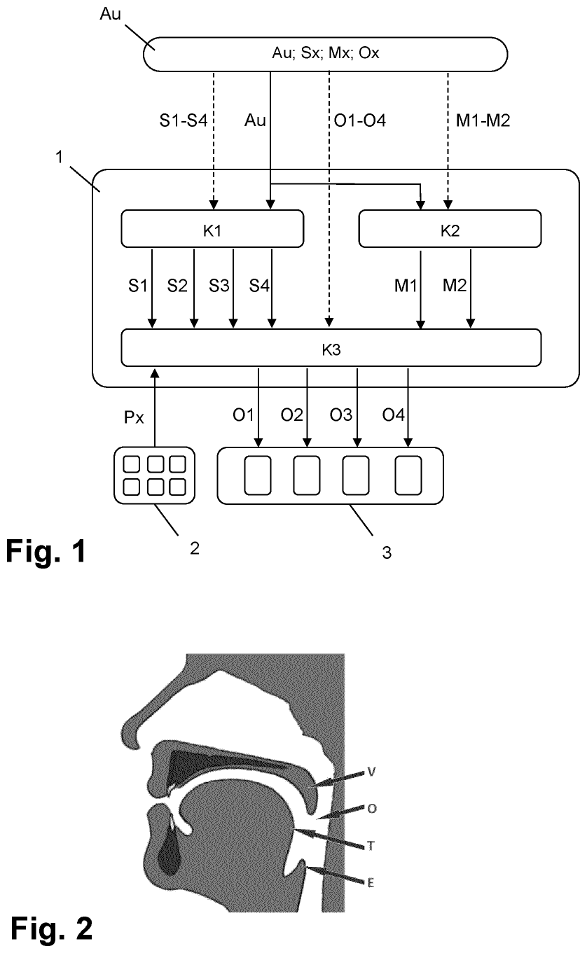

FIG. 1 schematically shows a classification system with a first and a second classifier to each of which a snoring-noise signal with a corresponding optional indicator is fed via an input interface, the output signals of the first and of the second classifier being fed to a third classifier for classification; the output signals of the third classifier which represent obstruction types are forwarded to a display unit via an output interface and are displayed there; additional snoring or patient data can be input via an input interface and fed to the third classifier for classification;

FIG. 2 shows a sectional lateral view of a patient's head including the neck-nose-throat area with the areas velopharynx, oropharynx, tongue base and epiglottis; and

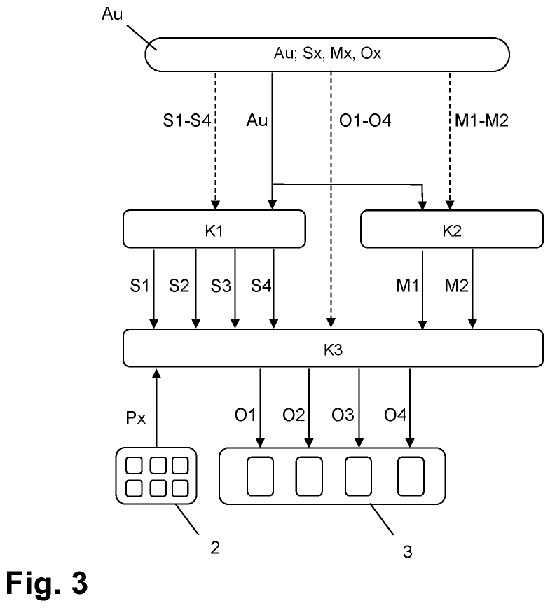

FIG. 3 shows a signal flow diagram of a method for determining the respective most probable obstruction type or of probabilities of the respective obstruction type from the snoring-noise signal, including the optional indicators for purposes of training of the first and the second classifier.

### DETAILED DESCRIPTION OF EMBODIMENTS

FIG. 1 schematically shows a possible embodiment of a classification system 1 for microprocessor-supported identification of obstruction types O1-O4 which can occur during sleep apnoea and are identified by classification system 1 from a snoring-noise signal Au to be examined. The classification system 1 comprises the following components:

A) an input interface for the respective snoring-noise signal Au which can have analog and/or digital inputs. For training the classification system 1, the snoring-noise signal Au has at least one additional indicator or a label with a type of snoring-noise origin S1-S4 and/or a mouth position M1-M2 which is assigned to the respective snoring-noise signal Au. Preferably, the snoring-noise signal Au also has an obstruction type O1-O4 as indicator which can be used for training the classification system 1. The input interface can generally also have an input for a keyboard, a button, an optical input or scanner or the like in order to record and forward the indicator(s) or labels.

B) a first classifier K1 adapted to learn in a training mode, when a first plurality of snoring-noise signals Au with a corresponding type of snoring-noise origin S1-S4 is input, such that in an identification mode, it can identify and output the most probable type of snoring-noise origin S1-S4 for a respective snoring-noise signal Au from a group of predefined types of snoring-noise origin S1-S4. Thus, the first classifier is a learning classifier. For clarity, if the snoring-noise signals Au of the training data were entered in the identification mode, the corresponding types of snoring-noise origin S1-S4 would be output correctly or at least on average with highest probability, with the preferred classifiers described above. If subsequently in the identification mode the snoring-noise signal Au to be examined is input, the most probable type of snoring-noise origin S1-S4 or the types of snoring-noise origin S1-S4 are determined as probability values and forwarded to a third classifier K3.
C) a second classifier K2 adapted to learn, when a second plurality of snoring-noise signals Au is input with a corresponding mouth position M1-M2 in the respective training mode, that in the identification mode, it identifies and outputs the corresponding most probable mouth position M1-M2 from a group of predefined mouth positions M1-M2 for the corresponding snoring-noise signal Au. The second classifier thus is a learning classifier as well. If subsequently in the identification mode the snoring-noise signal Au to be examined is input, the most probable mouth position M1-M2 or the mouth positions M1-M2 are determined as probability values and forwarded to the third classifier K3.
   D) the third classifier K3 which is adapted to identify in an identification mode, when the type of snoring-noise origin S1-S4 identified by the first classifier K1 and the mouth position M1-M2 identified by the second classifier K2 are input, the most probable obstruction type O1-O4 of a group of predefined obstruction types O1-O4 and to output it as an obstruction type signal.

Preferably, the third classifier K3 is adapted to learn in a training mode, when the type of snoring-noise origin S1-S4 identified by the first classifier K1, the mouth position M1-M2 identified by the second classifier K2 and an obstruction type O1-O4 are input, such that in the identification mode, it will identify, for the respective type of snoring-noise origin S1-S4 and the respective mouth position M1-M2, the input obstruction type O1-O4 as the most probable obstruction type O1-O4.

Preferably, the third classifier K3 is adapted to learn in a training mode, when the type of snoring-noise origin S1-S4 identified by the first classifier K1, the mouth position M1-M2 identified by the second classifier K2 and an obstruction type O1-O4 are input, such that it will recognize, in the identification mode, the input obstruction type O1-O4 as the most probable obstruction type O1-O4 for the respective type of snoring-noise origin S1-S4 and mouth position M1-M2.

If the snoring-noise signal Au to be examined is input in the identification mode, the types of snoring-noise origin S1-S4 identified by the first classifier K1 and the mouth positions M1-M2 identified by the second classifier K2 are assigned to the most probable obstruction type(s) O1-O4 with corresponding probability values. The third classifier K3 can also be a connection matrix which, as described above, performs a precisely defined probability assessment by means of input parameters such as at least the types of snoring-noise origin S1-S4 and the mouth positions M1-M2. During this process, the connection matrix can also be adapted, by means of an implemented or subordinated learning algorithm, such that the precisely predefined probability assessment is preferably further learned before an identification mode or during continuous identification in a training mode; and

E) an output interface 3 with a display for the obstruction type signal.

Preferably, the classification system 1 also has an input interface 2 by means of which the additional snoring and patient data Px can be input which are, for instance, taken into account by the third classifier K3 during classification of the respective obstruction type O1-O4.

For purposes of clarity, it is noted that by the type(s) of snoring-noise origin S1-S4, the mouth position(s) M1-M2 and the obstruction type(s) O1-O4, signals may be intended where they have signal properties.

Preferably, an identification precision is determined by means of annotated test data. Preferably, the test data are an independent part of the training data set which, however, was not used for training.

Preferably, the snoring-noise signal Au is a signal or a signal vector comprising a microphone or audio signal representing the snoring-noise signal and one or more characteristics signals. The microphone or audio signal representing the snoring-noise signal can be preprocessed in various ways, for instance by bandpass filtering or as known in the state of the art.

Alternatively preferably, the snoring-noise signal Au is a characteristics vector obtained from the audio signal by means of a characteristics extractor, consisting of at least one or more acoustic characteristics. The acoustic characteristics can for instance be a fundamental frequency, a harmonic-noise-ratio—HNR—, Mel-Frequency Cepstral Coefficient—MFCC— and/or others. The characteristics extractor preferably extracts instead of an individual value per characteristic which describes an entire time period of a snoring event, information on a chronological history of the acoustic characteristics which are preferably presented as static values. The static values are preferably an average value, a median value, a standard deviation and/or a Gauss distribution.

A method suitable for the classification system described above for microprocessor-supported identification of the obstruction types O1-O4 in case of sleep apnoea by classification of the recorded snoring-noise signal Au to be examined comprises the following steps:
A) training of a first classifier K1 by inputting at its input port a first plurality of snoring-noise signals Au to which a respective type of snoring-noise origin S1-S4 is assigned, for classification and output of the respective most probable type of snoring-noise origin S1-S4 in a respective identification mode, the respective type of snoring-noise origin S1-S4 originating from a first group of classes of the possible types of snoring-noise origin S1-S4;
B) training of a second classifier K1 by inputting at its input port a second plurality of snoring-noise signals Au to which a respective mouth position M1-M2 is assigned, for classification and output of the respective most probable mouth position M1-M2 in a respective identification mode, the respective mouth position M1-M2 originating from a second group of classes of the possible mouth positions M1-M2;
C) preferably training or matrix-shaped association of a third classifier K3 by inputting at its input port the types of snoring-noise origin S1-S4 and mouth positions M1-M2 identified above for classification in the corresponding identification mode and output of the most probable obstruction type O1-O4 in case of sleep apnoea, the respective obstruction type O1-O4 originating from a third group of classes of the obstruction types O1-O4; alternatively, the third classifier K3 can also be preprogrammed by a parameter input for classification of the most probable obstruction type O1-O4;
D) identifying, in the respective identification mode, the type of snoring-noise origin S1-S4 from the snoring-noise signal Au by means of the first classifier K1, the mouth position M1-M2 by means of the second classifier K2, and the resulting obstruction type O1-O4 by means of the third classifier K3; and
E) outputting the obstruction type O1-O4 for the snoring-noise signal Au to be examined, which was identified by means of the first K1, the second K2 and the third classifier K3, at an output interface 3.
FIG. 3 shows an example of the method described above as a signal flow diagram.

Preferably, the method described above also comprises the following, wherein training of the first classifier K1 and training of the second classifier K2 with a plurality of the snoring-noise signals Au take place separately from one another, wherein the first classifier K1 being trained and learning independently of the mouth position M1-M2 and the second classifier K2 independently of the type of snoring-noise origin S1-S4. Preferably, training and learning of the first K1 and the second classifier K2 take place with a time shift or simultaneously.

Preferably, the method described above also comprises the following, wherein training of the first classifier K1 and training of the second classifier K2 with another plurality of the snoring-noise signals Au together and simultaneously, the type of snoring-noise origin S1-S4 and the respective mouth position M1-M2 being assigned to the respective employed snoring-noise signal Au.

Preferably, the method described above also comprises the following, wherein in the identification mode, the respective types of snoring-noise origin S1-S4 are identified by the first classifier K1 and fed to the third classifier K3.

Preferably, the method described above also comprises the following, wherein in the identification mode, the respective mouth positions M1-M2 are identified by the second classifier K2 and fed to the third classifier K3 for identification of the obstruction type O1-O4.

Preferably, the method described above also comprises the following, wherein in the identification mode, the respective obstruction type O1-O4 is identified by the third classifier K3 from the respective types of snoring-noise origin S1-S4 and mouth positions M1-M2, with indication of a corresponding probability.

Preferably, the method described above also comprises the following, wherein the first group of the types of snoring-noise origin S1-S4 comprising the following classes: velopharynx (V), oropharynx (O), tongue base area (T) and/or epiglottis area (E). Preferably, the respective type of snoring-noise origin S1-S4 also includes an orientation of the vibration, which can for instance be a lateral or a circular vibration.

Preferably, the second group of mouth positions comprises the following mouth positions: mouth open, mouth closed. Alternatively preferably, the second group of mouth positions can include more than two mouth positions with intermediate positions.

Preferably, the method described above is adapted such that in addition to the respective type of snoring-noise origin S1-S4 and the respective mouth position M1-M2, additional snoring or patient data Px associated with the snorer are fed to the third classifier K3, which data are taken into account and evaluated by the third classifier K3 during training and/or identification of the obstruction type O1-O4.

Preferably, the snoring or patient data Px comprise at least one of the following parameters: body mass index, apnoea hypopnoea index, size of tonsils, size of tongue, Friedman score, time of snoring, duration of sleep.

For purposes of clarity, it is noted that the indefinite article “a” in connection with an object does not limit the number of objects to exactly “one”, but that “at least one” is intended. This shall apply to all indefinite articles for example “a” etc.

For purposes of clarity, the terms “first”, “second”, “third” etc. as used herein are only employed to distinguish different pluralities, elements and/or components. Therefore, for instance, a first plurality can also be termed as second plurality, and consequently the second plurality can also be termed first plurality without deviating from the teachings of the present invention.

It is understood that instead of the two or four classes mentioned herein of types of snoring-noise origin, mouth positions and obstruction types, other pluralities can be used or detected as well.

The reference signs indicated in the Claims are only for better comprehensibility and do not limit the Claims in any way to the embodiments shown in the Figures.

### LIST OF REFERENCE SIGNS

1 classification system

2 input interface

3 output interface

Au snoring-noise signal

Sx, S1-S4 type of snoring-noise origin

Mx, M1, M2 mouth position

Ox, O1-O4 obstruction type

K1 first classifier

K2 second classifier

K3 third classifier

Px snoring or patient data

V velopharynx

O oropharynx

T area of tongue base

E area of epiglottis

1. Classification system for microprocessor-supported identification of obstruction types in sleep apnoea by means of appropriate classification of a snoring-noise signal to be examined, comprising:
a) an input interface for the respective snoring-noise signal;
b) a first classifier adapted to learn in a training mode, when a first plurality of snoring-noise signals is input with a corresponding type of snoring-noise origin, such that in an identification mode, it identifies and outputs the most probable type of snoring-noise origin for a particular snoring-noise signal from a group of predefined types of snoring-noise origin;
c) a second classifier adapted to learn in a training mode, when a second plurality of snoring-noise signals is input with a corresponding mouth position, such that in an identification mode, it identifies and outputs the most probable mouth position for a particular snoring-noise signal from a group of predefined mouth positions;
d) a third classifier adapted to identify in an identification mode, when the type of snoring-noise origin identified by the first classifier and the mouth position identified by the second classifier are input, from a group of predefined obstruction types the most probable obstruction type and output it as an obstruction type signal; and
e) an output interface to a display for the obstruction-type signal.

2. The classification system according to claim 1, the first classifier and the second classifier being adapted such that the respective training of the first and of the second classifier with a plurality of snoring-noise signals can be performed separately from one another, with the first classifier training and learning independently of the mouth position and the second classifier independently of the type of snoring-noise origin.

3. The classification system according to claim 1, the first and the second classifier being adapted such that the respective training of the first and the second classifier with an additional plurality of snoring-noise signals takes place together and simultaneously, the respective snoring-noise signal used including the respective type of snoring-noise origin and the respective mouth position as corresponding information.

4. The classification system according to claim 1, the first classifier being adapted to identify, indicate and forward to the third classifier, in the identification mode, the respective type of snoring-noise origin with a respective probability.

5. The classification system according to claim 1, the second classifier being adapted to identify, indicate and forward to the third classifier, in the identification mode, the respective mouth position with a respective probability.

6. The classification system according to claim 1, the third classifier being adapted to learn in a training mode, when the type of snoring-noise origin identified by the first classifier, the mouth position identified by the second classifier and an obstruction type are input, such that in the identification mode, it identifies the input obstruction type as the most probable obstruction type with the respective type of snoring-noise origin and the respective mouth position.

7. The classification system according to claim 1, the third classifier being adapted, in an identification mode, to identify, indicate and forward to the output interface the respective obstruction type with a respective probability.

8. The classification system according to claim 1, the third classifier being adapted to identify, in addition to the respective type of snoring-noise origin and the respective mouth position, other snoring or patient data associated with the snorer via an input interface and, in the training mode and/or in the identification mode, take them into account as parameters or parameter signals in classifying the obstruction type.

9. The classification system according claim 8, the snoring or patient data comprising at least one of the following parameters: body mass index, apnoea hypopnoea index, size of tonsils, size of tongue, Friedman score, time of snoring, duration of sleep.

10. The classification system according to claim 1, the first classifier being based on one of the following methods of machine learning: Support Vector Machine—SVM—, Naïve-Bayes-System, Least Mean Square method, k-Nearest Neighbours method—k-NN—, Linear Discriminant Analysis—LDA—, Random Forests method—RF—, Extreme Learning Machine—ELM—, Multilayer Perceptron—MLP—, Deep Neural Network—DNN—, logistic regression.

11. The classification system according to claim 1, wherein
the second classifier is based on one of the following methods of machine learning: Support Vector Machine—SVM—, Naïve-Bayes-System, Least Mean Square method, k-Nearest Neighbours method—k-NN—, Linear Discriminant Analysis—LDA—, Random Forests method—RF—, Extreme Learning Machine —ELM—, Multilayer Perceptron—MLP—, Deep Neural Network—DNN—, logistic regression.

12. The classification system according to claim 1, wherein
the third classifier is based on one of the following methods of machine learning: Support Vector Machine—SVM—, Naïve-Bayes-System, Least Mean Square method, k-Nearest Neighbours method—k-NN—, Linear Discriminant Analysis—LDA—, Random Forests method—RF—, Extreme Learning Machine—ELM—, Multilayer Perceptron—MLP—, Deep Neural Network—DNN—, logistic regression.

13. The classification system according to claim 1, wherein
the third classifier is based on a matrix probability assessment of a first input vector of the types of snoring-noise origin and of at least one second input vector of the mouth positions, whose summary probabilities in turn result in the various obstruction types and their probabilities.

14. A method for a microprocessor-supported identification of obstruction types in case of sleep apnoea by classification of a recorded snoring-noise signal to be examined, comprising the following steps:
A) training of a first classifier by inputting at its input port a first plurality of snoring-noise signals to which a respective type of snoring-noise origin is assigned, for classification and output of a respective most probable type of snoring-noise origin in a respective identification mode, the respective type of snoring-noise origin originating from a first group of classes of possible types of snoring-noise origins;
B) training of a second classifier by inputting at its input port a second plurality of snoring-noise signals to which a respective mouth position is assigned, for classification and output of a respective most probable mouth position in a respective identification mode, the respective mouth position originating from a second group of classes of possible mouth positions;
C) either training or matrix-shaped association of a third classifier by inputting at its input port the types of snoring-noise origin and mouth positions identified above for classification in the corresponding identification mode and output of a most probable obstruction type in case of sleep apnoea, the respective obstruction type originating from a third group of classes of obstruction types,
or alternatively using the third classifier being preprogrammed by a parameter input for classification of the most probable obstruction type;
D) identifying, in a respective identification mode, the type of snoring-noise origin from the snoring-noise signal by means of the first classifier, the mouth position by means of the second classifier, and the resulting obstruction type by means of the third classifier; and
E) outputting the obstruction type for the snoring-noise signal to be examined, which was identified by means of the first, the second and the third classifier, at an output interface.

15. The method according to claim 14, wherein the training of the first classifier and the training of the second classifier with a plurality of the snoring-noise signals take place separately from one another, wherein the first classifier is trained and learns independently of the mouth position and the second classifier is trained independently of the type of snoring-noise origin.

16. The method according to claim 15, wherein the training and learning of the first and the second classifier take place with a time shift.

17. The method according to claim 15, wherein the training and learning of the first and the second classifier take place simultaneously.

18. The method according to claim 14, wherein the training of the first classifier and the training of the second classifier with another plurality of the snoring-noise signals take place together and simultaneously, wherein the type of snoring-noise origin and the respective mouth position being assigned to the respective employed snoring-noise signal.

19. The method according to claim 14, wherein in the identification mode, the respective types of snoring-noise origin are identified by the first classifier with respective probability values and fed to the third classifier.

20. The method according to claim 14, wherein in the identification mode, the respective mouth positions are identified by the second classifier with respective probability values and fed to the third classifier for identification of the obstruction type.

21. The method according to claim 14, wherein in the identification mode, the respective obstruction type is identified by the third classifier from the respective types of snoring-noise origin and mouth positions, with indication of a corresponding probability.

22. The method according to claim 14, wherein the first group of the types of snoring-noise origin comprise the following classes: velopharynx, oropharynx, tongue base area and/or epiglottis area.

23. The method according to claim 22, wherein the respective type of snoring-noise origin includes an orientation of the vibration, which is a lateral or a circular vibration.

24. The method according to claim 14, wherein the second group of mouth positions comprises the following mouth positions: mouth open, mouth closed.

25. The method according to claim 14, wherein the second group of mouth positions include mouth positions:
mouth open, mouth closed, and intermediate mouth positions.

26. The method according to claim 14, wherein in addition to the respective type of snoring-noise origin and the respective mouth position additional snoring or patient data associated with the snorer are fed to the third classifier, which snoring or patient data are taken into account and evaluated by the third classifier during training and/or identification of the obstruction type.

27. The method according to claim 14, wherein the snoring or patient data comprise at least one of the following parameters: body mass index, apnoea hypopnoea index, size of tonsils, size of tongue, Friedman score, time of snoring, duration of sleep.
